(19) United States
(12) Patent Application Publication
(10) Pub. No.: US 2014/0371776 A1
(43) Pub. Date: Dec. 18, 2014

(54) INTERNAL NASAL SEPARATOR

(71) Applicant: Manuel MOYA VICENTE, SEVILLA (ES)

(72) Inventor: Manuel MOYA VICENTE, SEVILLA (ES)

(21) Appl. No.: 14/292,971

(22) Filed: Jun. 2, 2014

(30) Foreign Application Priority Data
Jun. 14, 2013 (ES) U201330750

(51) Int. Cl. A61F 5/08

(52) U.S. Cl. CPC A61F 5/08
USPC 606/198

(57) ABSTRACT

The internal nasal separator is to be used inside a user's nostrils. It comprises a pair of arms (1), a curved connector (2) used to join the arms (1), which carries out spring functions and a pair of ellipsoid shape end devices (3) built into the ends of both arms (1). The arms (1) are closed together the right distance towards the inside and then the end devices (3) are introduced inside the nostrils towards the deep end to be released and left to rest on the inner walls of the nostrils. This prevents nostrils from contracting upon breathing and keeping the nostrils open.

### OBJECT OF THE INVENTION

The present invention describes a nasal separator device for use inside the nostrils, with the aim of expanding them and preventing them from contracting during breathing, in order to thus facilitate user breathing.

### TECHNICAL PROBLEM TO BE SOLVED AND BACKGROUND OF THE INVENTION

In the current state of the art, a wide range of devices used to widen the nasal passages are known.

The devices that currently exist in the state of the art may be divided into two main groups, according to those used externally and those used inside the nostrils.

The devices used outside the nose, despite having the same aim as those used inside the nostrils, do not have much in common with the invention described herein, therefore we shall not go into further detail as regards the characteristics thereof. In patent documents ES-2178151_T3 and ES-2231938_T3, devices of this kind are described.

Despite there being a high number of existing developments related to the dilation of the nostrils, widespread interest in developing systems for improving breathing by means of mechanically manipulating the nostrils continues to exist.

The high number of users who consume this kind of product, the swift way in which situations that these products might alleviate arise, for example catarrh, allergies, etc., and the ease of application are reasons why the production and distribution of this product are considered financially attractive.

As far as those devices used inside the nostrils are concerned, documents closer to the state of the art may be cited, for example those described in patent ES-2251 013_T3 and utility model ES-1067455_U.

Document ES-2251 013_T3 describes a double U-shaped device, joined by one of the ends of the arms. The device is fitted on the bridge of the nose with the ends introduced in each nostril. These ends are wider at the end, thus making it possible to widen the entire nasal cavity.

In document ES-1067455_U, a device, also U-shaped, is described, the ends of which have been extended and bent, in such a way that the U inversely surrounds the nose from the outside and each end is introduced into each of the nostrils.

The device object of the present invention uses a mechanism designed to operate using a spring to open the nostrils, thus achieving the aim sought by means of a very simple method.

### DESCRIPTION OF THE INVENTION

The present invention describes a nasal separator for use inside the user's nostrils.

The nasal separator is composed of a pair of arms, a curved connector which connects the arms and which carries out the spring functions and a pair of end devices built into the end of both arms.

The end devices are introduced into the nostrils, by squeezing the arms the right distance towards the inside and moved towards the deep end to be subsequently released and left to rest on the inner walls of the nostrils, in order to thus successfully leave them open and prevent them from contracting upon breathing.

The connector is a fold formed by both arms of the nasal separator and may form a rotation angle ranging from 90° to several turns.

The arms form an aperture angle of 45° when they are in resting position.

### BRIEF DESCRIPTION OF THE DRAWINGS

In order to complement the invention described herein, with the aim of facilitating a better understanding of the characteristics thereof, in accordance with a preferred embodiment of the same, a number of drawings are attached, which provide a non-limiting illustration of the invention, in a series of figures facilitating understanding of the device.

FIG. 1 is a perspective view of the device.

FIG. 2 is a plan view of the device when being used in the nose.

Below is a list of reference numbers employed in the figures:

1. Arms.

2. Connector.

3. End devices.

4. Aperture angle.

### DESCRIPTION OF A PREFERRED EMBODIMENT OF THE INVENTION

The present invention refers to a nasal separator, which is introduced into the nose with the aim of separating the internal walls of the nostrils and preventing them from contracting upon breathing.

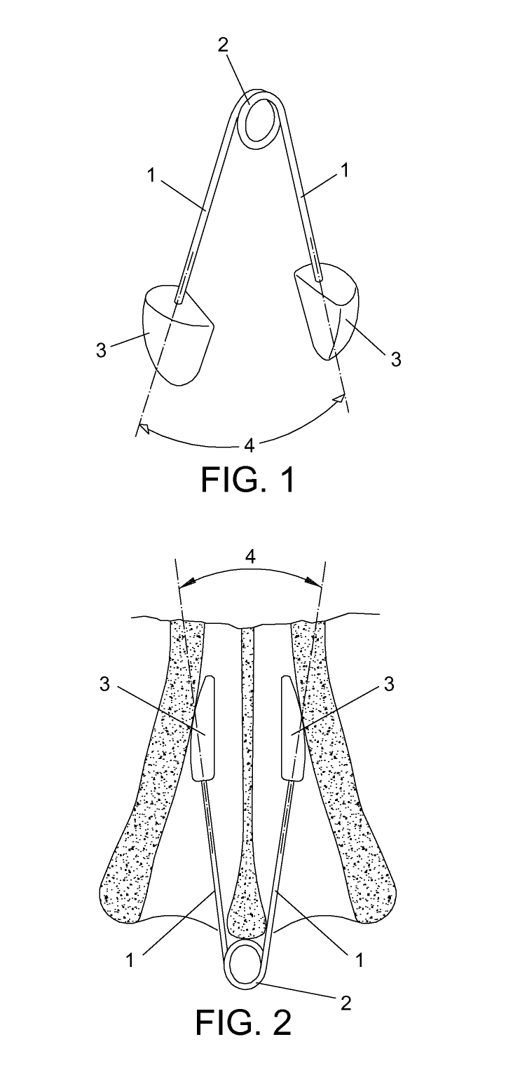

As can be seen in FIG. 1, the nasal separator is formed by a main body formed by two arms (1), one of their ends joined by means of a connector (2), the other ends terminated with end devices (3).

The connector (2) is the area in which the arms (1) are joined and is an extension of the same, where a fold is formed that may extend from a few degrees to several turns. This fold generates an aperture angle (5) in the arms (1), which may be any value, a standard value being 45°.

As can be seen in FIG. 2, the arms (1) are practically parallel in the deep end position of the nasal separator located inside the nostrils. The force generated by the end devices (3) on the internal sidewalls of the nostrils is that due to the flexion suffered by the connector (2) when the arms (1) are closed from the aperture angle (5) in resting position. The aperture angle (5) is therefore directly related to said force.

The end device (3) serves to provide a support surface for the arms (1) of the nasal separator, so that they apply pressure to the internal walls of the nostrils and, upon pressure being applied thereon, the end device (3), given the spring effect of the connector (2), forces the nostril walls to separate, thus preventing them from contracting.

The main body is formed by a rigid, elastic material. Although this may be metal, plastic or even some kind of rubber providing the necessary rigidity is preferably used, considering that the force required is not very high.

In the figures showing this preferred embodiment, the nasal separator is shown as being made from a filiform metal material, which has been turned several times, leaving the arms (1) with an aperture angle (5) of 45° in resting position. This angle determines the pressure exerted by the end devices (3) on the internal walls of the nostrils once the nasal separator has been fitted, the value of 45° being merely for simplicity purposes, it being possible for any value to be employed. In this case, the number of turns that form the connector (2) depends directly on the closing force of the arms (1). Another factor that has a decisive influence upon the force to be applied to the nostrils is the material, this being a direct result of the material's elasticity module.

The nasal separator may be produced in two sizes that cover the entire market, one of these sizes being aimed on adults and the other on children. The opening force of the two end devices (3) on the inner walls of the nostrils, as described, depends on the aperture angle (4) of the arms (1) and also on characteristics such as the length of the arms (1) or the elasticity module of the material used. With the first two characteristics, a variety of force levels may be achieved in the arms (1), although a variety of different designs is also required. In contrast, with the same design, only changing the manufacturing material, different elastic modules provide different levels of force, thus meaning a range of different force levels may be obtained in the arms (1) using just one design.

The present invention is not limited to the embodiment described herein.

Other configurations may be carried out by experts in the field based on the present description. As a result, the scope of the invention is defined by the following claims.

1. Internal nasal separator for use inside a user's nostrils, characterised in that it comprises:
a pair of arms (1),
a curved connector (2) used to join the arms (1), which carries out spring functions,
a pair of ellipsoid shape end devices (3) built into the ends of both arms (1),
where the end devices (3) are introduced inside the nostrils by squeezing the arms (1) the right distance towards the inside, moving towards the deep end in order to later be released and left to rest on the inner walls of the nostrils, in order to thus keep them open and prevent them from contracting upon breathing.

2. Internal nasal separator according to claim 1, characterised in that the connector (2) is a fold formed by both arms (1) with a rotation angle going from 90° up to several turns.

3. Internal nasal separator according to claim 2, characterised in that the aperture angle (5) is 45°.
